(19) United States
(12) Patent Application Publication
(10) Pub. No.: US 2024/0300908 A1
(43) Pub. Date: Sep. 12, 2024

(54) CHIRAL 3-SULFINYL BENZOIC ACIDS

(71) Applicant: BAYER AKTIENGESELLSCHAFT, LEVERKUSEN (DE)

(72) Inventors: Sergii PAZENOK, Leverkusen (DE); Eike Kevin HEILMANN, Leverkusen (DE); Heiko SCHIRMER, Leverkusen (DE); Klaus-UIrich SCHIFFER, Leverkusen (DE); Kai LOVIS, Leverkusen (DE); Laura KQIKU, Leverkusen (DE)

(21) Appl. No.: 18/570,473

(22) PCT Filed: Jul. 4, 2022
(86) PCT No.: PCT/EP2022/068443

§ 371 Date: Dec. 14, 2023

(30) Foreign Application Priority Data
Jul. 8, 2021 (EP) 21184514.4

(51) Int. Cl. C07D 271/113

(52) U.S. Cl. CPC C07D 271/113

(57) ABSTRACT

Chiral 3-sulfinylbenzoic acids of the respective absolute configuration given in formulae (I-R) and (I-S) are described as precursors for preparation of herbicidal compounds.

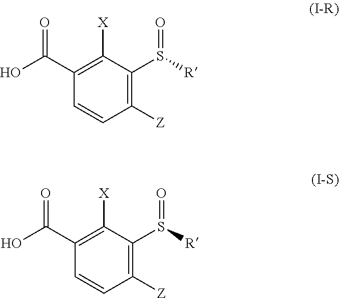

In formulae (I-R) and (I-S). X. Z and R′ represent radicals such as alkyl, cycloalkyl, haloalkyl and halogen.

The invention relates to chiral 3-sulfinylbenzoic acids, to the use thereof and to a process for preparing chiral N-(1,2,5-oxadiazol-3-yl)-, N-(1,3,4-oxadiazol-2-yl)-, N-(tetrazol-5-yl)- and N-(triazol-5-yl)phenylcarboxamides.

WO 2021/078174 A1 discloses herbicidally active chiral N-(1,2,5-oxadiazol-3-yl)-, N-(1,3,4-oxadiazol-2-yl)-, N-(tetrazol-5-yl)- and N-(triazol-5-yl)phenylcarboxamides. EP 21162218 likewise discloses herbicidally active chiral N-(1,3,4-oxadiazol-2-yl)phenylcarboxamides. The herbicidally active chiral compounds described therein bear a chiral sulfinyl group in the 3 position of the phenyl ring. These compounds are prepared in a complex manner by enantiomeric separation of the N-(1,2,5-oxadiazol-3-yl)-, N-(1,3,4-oxadiazol-2-yl)-, N-(tetrazol-5-yl)- and N-(triazol-5-yl)phenylcarboxamides.

It was an object of the present invention to overcome the disadvantages known from the prior art.

The present invention provides chiral 3-sulfinylbenzoic acids of the respective absolute configuration given in formulae (I-R) and (I-S)

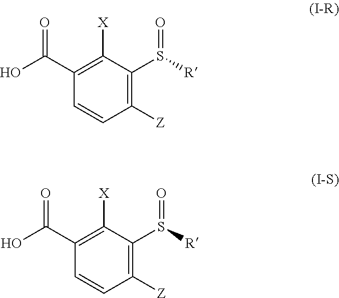

in which the substituents are defined as follows:
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR^{a}, S(O)ₙR^{b} or (C₁-C₆)-alkyl-OR^{a},
Z is halogen, (C₁-C₆)-alkyl, halo-(C₁-C₆)-alkyl, (C₃-C₆)-cycloalkyl or S(O)ₙR^{b},
R^{a} is (C₁-C₆)-alkyl or (C₃-C₆)-cycloalkyl.
R^{b} is (C₁-C₆)-alkyl or (C₃-C₆)-cycloalkyl.
n is 0, 1 or 2.

Compounds of the invention are those of the general formula (I-S) which, according to the Cahn-Ingold-Prelog rules, are in the S configuration, provided that R′ has a lower priority than the phenyl ring. This is true, for example, of compounds of the general formula (I) in which R′ is methyl or cyclopropyl. Further compounds of the invention are those of the general formula (I) which, according to the Cahn-Ingold-Prelog rules, are in the R configuration, provided that R′ has a higher priority than the phenyl ring. This is true, for example, of compounds of the general formula (I) in which R′ is methoxymethyl.

In the formulae (I-R) and (I-S) and all the formulae which follow, alkyl radicals having more than two carbon atoms may be straight-chain or branched. Alkyl radicals are, for example, methyl, ethyl, n-propyl or isopropyl, n-, iso-, t- or 2-butyl, pentyls, hexyls such as n-hexyl, isohexyl and 1,3-dimethylbutyl. Cycloalkyl is a carbocyclic saturated ring system having three to six carbon atoms, for example cyclopropyl, cyclobutyl, cyclopentyl or cyclohexyl. Halogen-substituted alkyl denotes straight-chain or branched alkyl groups where some or all of the hydrogen atoms in these groups may be replaced by halogen atoms, e.g. C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl.

Halogen represents fluorine, chlorine, bromine or iodine.

If a group is polysubstituted by radicals, this should be understood to mean that this group is substituted by one or more identical or different radicals selected from the radicals mentioned.

Preference is given to compounds of the general formulae (I-R) and (I-S) in which
X is F, Cl, Br, methyl, ethyl, i-Pr, c-Pr, OMe, SMe, SEt, CH₂OMe or CF₃,
R′ is methyl, ethyl, c-Pr, CH₂-cPr, CH₂CH₂OMe, c-Pr, CH₂-cPr or CH₂CH₂OMe.
Z is F, Cl, Br, I, methyl, ethyl, c-Pr, i-Pr, SMe, S(O)Me, S(O)₂Me, S(O)₂Et, CF₃, C₂F₅ or CHF₂.

Particular preference is given to compounds of the general formulae (I-R) and (I-S) in which
X is F, Cl, Br, methyl, ethyl, c-Pr, OMe, SMe, SEt, CH₂OMe or CF₃.
R′ is Me, Et, c-Pr, CH₂-cPr or CH₂CH₂OMe.
Z is Cl, Br, methyl, ethyl, c-Pr, i-Pr, S(O)₂Me, S(O)₂Et, CF₃, C₂F₅ or CHF₂.

Very particular preference is given to compounds of the general formulae (I-R) and (I-S) in which
X is Cl or methyl.
R′ is methyl or c-Pr.
Z is CF₃ or CHF₂.

In all the formulae specified hereinafter, the substituents and symbols have the same meaning as described in formulae (I-R) and (I-S), unless defined differently. OMe means O-methyl; SMe means S-methyl; SEt means S-ethyl; CH₂OMe means CH₂O-methyl; i-Pr means isopropyl; c-Pr means cyclopropyl.

Inventive compounds of the general formulae (I-R) and (I-S) can be prepared, for example, by processes described hereinafter from the respective racemic compounds (I-rac). These processes likewise form part of the subject-matter of the present invention.

The racemic compounds (I-rac) and the preparation thereof are known in principle, for example, from WO 2021/078174 A1 and WO 2012/126932 A1. The racemic compounds (I-rac) are reacted with an enantiomerically pure amine of the general formula (II); under suitable conditions, only one of the two possible diastereomeric salts (III-dR) and (III-dS) crystallizes out and can be separated off for further workup. The other diastereomeric salt can be isolated from the mother liquor.

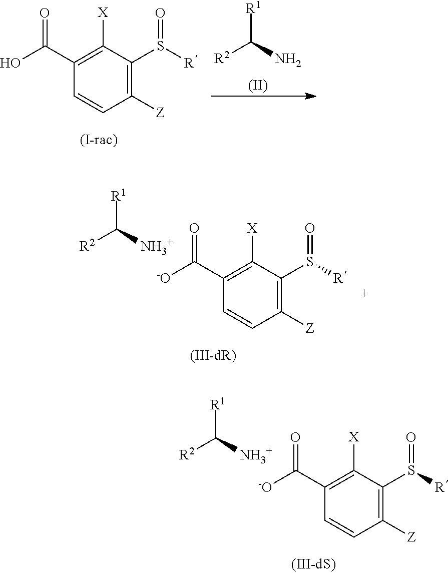

The crystallization may take place in various suitable solvents or solvent mixtures, using methanol, methanol/water (1:1 to 10:1), ethanol/water (1:1 to 10:1), isopropanol, preferably isopropanol/water (range of 1:1 to 10:1), acetone/water (1:1 to 20:1), ethyl acetate, THF. THF/water (3:1 to 20:1) or toluene. The salt crystals obtained are separated from the mother liquor by filtration using the known methods and washed with the solvent or solvent mixture used and dried under reduced pressure. In a further reaction step, the isolated diastereomeric compounds of the general formula (III-dR) or (III-dS) are then mixed with water at a temperature of 0° C. to 20° C. optionally in the presence of organic solvents such as methanol, ethanol, isopropanol. THF, acetone etc., and admixed with a strong acid such as HCl or H₂SO₄ in order to reach a pH of 1-2. The enantiomerically pure compounds of the general formula (I-R) or (I-S) precipitate out and are separated from the mother liquor by filtration, washed and dried under reduced pressure.

The compounds of the formula (I-rac) and the amine of the formula (II) are typically used in equimolar amounts. This step is normally executed at room temperature.

Suitable chiral amines are a multitude of commercially available amines of the formula (II), for example those in which
R¹ is methyl, ethyl, n-propyl, isopropyl, n-butyl, isobutyl, and
R² is hydroxymethyl, phenyl, 4-methylphenyl.

For example, the following amines of the formula (II) are of good suitability:
(S)-(−)-α,4-dimethylbenzylamine (CAS No. 27298-98-2),
(R)-(−)-3-methyl-2-phenylbutylamine (CAS No. 67152-35-6).
(S)-(+)-2-amino-3-methyl-1-butanol (CAS No. 2026-48-4).

Preference is given to (S)-(−)-α,4-dimethylbenzylamine (CAS No. 27298-98-2).

The inventive 3-sulfinylbenzoic acids of the formulae (I-R) and (I-S) are obtained in the aforementioned process generally with an enantiomeric excess (ee) of at least 94%, often even at least 99%.

3-Sulfinylbenzoic acids of the formulae (I-R) and (I-S) with an enantiomeric excess (ee) of at least 94% are preferred. 3-Sulfinylbenzoic acids of the formulae (I-R) and (I-S) with an enantiomeric excess (ee) of at least 99% are particularly preferred.

Inventive 3-sulfinylbenzoic acids of the general formula (I-S) are of particularly good suitability for preparation of herbicidally active compounds as described in EP 21162218.

The present invention thus further provides a process for preparing N-(1,3,4-oxadiazol-2-yl)phenylcarboxamides having the absolute configuration given in formula (I*) by reacting 2-amino-1,3,4-oxadiazoles of the general formula (III) with inventive 3-sulfinylbenzoic acids of the general formula (I-S),

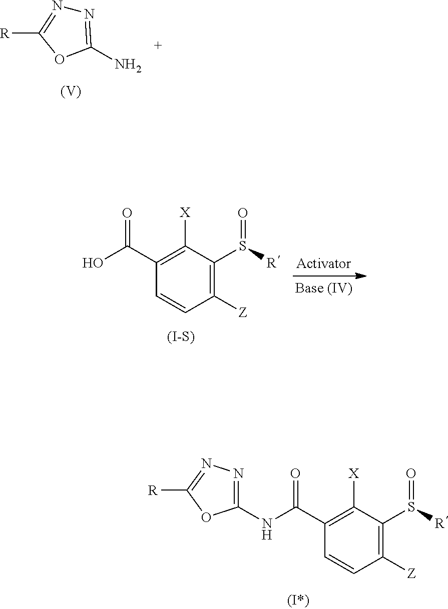

characterized in that it is performed
   a) in the presence of an activating reagent (activator) from the group consisting of thionyl chloride, phosgene, diphosgene, mesyl chloride, tosyl chloride, POCl₃, PCl₅, oxalyl chloride and C₁-C₈-alkyl-OC(O)Cl, and
   b) in the presence of a base of the general formula (IV),

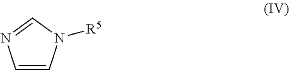

and
c) in which the substituents are as defined below:
R is hydrogen, (C₁-C₆)-alkyl, (C₃-C₇)-cycloalkyl, methoxymethyl or methoxyethyl,
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR¹, S(O)ₙR² or (C₁-C₆)-alkyl-OR¹,
Z is halogen, (C₁-C₆)-alkyl, halo-(C₁-C₆)-alkyl, (C₃-C₆)-cycloalkyl or S(O)ₙR²,
R¹ is (C₁-C₆)-alkyl or (C₃-C₆)-cycloalkyl,
R² is (C₁-C₆)-alkyl or (C₃-C₆)-cycloalkyl,
R⁵ is C₁-C₁₂-alkyl or phenyl,
n is 0, 1 or 2.

Inventive 3-sulfinylbenzoic acids of the general formula (I-R) are of particularly good suitability for preparation of herbicidally active compounds as described in WO 2021/078174 A1. The present invention thus further provides a process for preparing N-(1,3,4-oxadiazol-2-yl)phenylcarboxamides having the absolute configuration given in formula (I**) by reacting 2-amino-1,3,4-oxadiazoles of the general formula (V) with inventive 3-sulfinylbenzoic acids of the general formula (I-R),

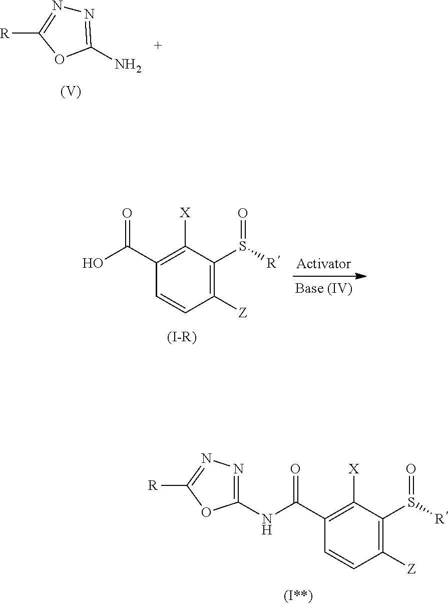

characterized in that it is performed
   a) in the presence of an activating reagent (activator) from the group consisting of thionyl chloride, phosgene, diphosgene, mesyl chloride, tosyl chloride, POCl₃, PCl₅, oxalyl chloride and C₁-C₈-alkyl-OC(O)Cl, and
   b) in the presence of a base of the general formula (IV),

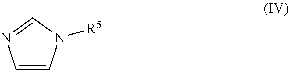

and
c) in which the substituents are as defined below:
R is hydrogen, (C₁-C₆)-alkyl, (C₃-C₇)-cycloalkyl, methoxymethyl or methoxyethyl,
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR¹, S(O)ₙR² or (C₁-C₆)-alkyl-OR¹,
Z is halogen, (C₁-C₆)-alkyl, halo-(C₁-C₆)-alkyl, (C₃-C₆)-cycloalkyl or S(O)ₙR²,
R¹ is (C₁-C₆)-alkyl or (C₃-C₆)-cycloalkyl,
R² is (C₁-C₆)-alkyl or (C₃-C₆)-cycloalkyl,
R⁵ is C₁-C₁₂-alkyl or phenyl,
n is 0, 1 or 2.

In the two above-described processes for preparing compounds of the formula (I*) from compounds of the formulae (V) and (I-S) or compounds of the formula (I**) from compounds of the formulae (V) and (I-R), the radicals are preferably as follows:
R is hydrogen or methyl,
X is F, Cl, Br, methyl, ethyl, i-Pr, c-Pr, OMe, SMe, SEt, CH₂OMe or CF₃,
R′ is methyl, ethyl, c-Pr, CH₂-cPr, CH₂CH₂OMe, c-Pr, CH₂-cPr or CH₂CH₂OMe,
Z is F, Cl, Br, I, methyl, ethyl, c-Pr, i-Pr, SMe, S(O)Me, S(O)₂Me, S(O)₂Et, CF₃, C₂F₅ or CHF₂;
more preferably:
R is hydrogen or methyl,
X is F, Cl, Br, methyl, ethyl, c-Pr, OMe, SMe, SEt, CH₂OMe or CF₃,
R′ is Me, Et, c-Pr, CH₂-cPr or CH₂CH₂OMe,
Z is C₁, Br, methyl, ethyl, c-Pr, i-Pr, S(O)₂Me, S(O)₂Et, CF₃, C₂F₅ or CHF₂;
very particularly:
R is hydrogen or methyl.
X is Cl or methyl.
R′ is methyl or c-Pr.
Z is CF₃ or CHF₂.

In the two above-described processes for preparing compounds of the formula (I*) from compounds of the formulae (V) and (I-S) or compounds of the formula (I**) from compounds of the formulae (V) and (I-R), the compounds of the formulae (V) and (I-S) or (V) and (I-R) are typically used in a molar ratio of 0.8 to 1.5. The compound of the formula (V) is preferably used with an excess of 10% relative to the compound of the formula (I-S) or (I-R).

The activator and the compounds of the formula (I-S) or (I-R) are typically used in a molar ratio of 0.5 to 3, preferably of 1 to 2, more preferably of 1.2 to 1.9.

The activator used is preferably thionyl chloride, phosgene or diphosgene, more preferably thionyl chloride.

The base of the formula (IV) and the compounds of the formula (I-S) or (I-R) are typically used in a molar ratio of 0.5 to 10, preferably of 1 to 3, more preferably of 1 to 2.5.

The two aforementioned processes of the invention for preparing the compounds of the formulae (I*) and (I**) are generally conducted in a solvent. Suitable solvents are inert organic solvents, preferably aliphatic, alicyclic or aromatic hydrocarbons such as petroleum ether, hexane, heptane, cyclohexane, methylcyclohexane, benzene, toluene, xylene and decalin; halogenated hydrocarbons such as chlorobenzene, dichlorobenzene, dichloromethane, chloroform, tetrachloromethane, dichloroethane and trichloroethane; esters such as ethyl acetate and isopropyl acetate; ethers such as diethyl ether, diisopropyl ether, methyl tert-butyl ether, methyl tert-amyl ether, dioxane, tetrahydrofuran, 1,2-dimethoxyethane, 1,2-diethoxyethane and anisole; ketones such as acetone, butanone, methyl isobutyl ketone and cyclohexanone; nitriles such as acetonitrile, propionitrile, n- or isobutyronitrile and benzonitrile; amides such as N,N-dimethylformamide, N,N-dimethylacetamide, N-methylformanilide, N-methylpyrrolidone and hexamethylphosphoramide; pyridines such as 2-methylpyridine, 3-methylpyridine, 4-methylpyridine, 2,3-dimethylpyridine, 2-methyl-5-ethylpyridine, 2,6-dimethylpyridine, 2,4-dimethylpyridine, 3,4-dimethylpyridine and 2,4,6-trimethylpyridine. Mixtures of the abovementioned solvents are also suitable.

The solvent used is preferably tetrahydrofuran, acetonitrile, 3-methylpyridine or 2-methyl-5-ethylpyridine. Particular preference is given to 3-methylpyridine.

These processes are typically conducted within a temperature range from −5° to 50° C. preferably 0° to 25° C.

These processes are typically conducted in such a way that the activator is slowly added dropwise with stirring, or introduced in the case of phosgene, to an initial charge of the compounds of the formulae (III), (I-S) and (IV) in a solvent. The progress of the reaction can be monitored by HPLC. The reaction generally goes to completion after 10 to 20 hours.

After the reaction is complete, the reaction mixture is cooled and the product generally precipitates out virtually quantitatively. Alternatively, the reaction mixture can be diluted with a polar solvent such as water or alcohols such as isopropanol. The reaction product of the formula (I*) or (I**) is obtained in high purity and can be purified further if required. It is particularly advantageous to add water to the reaction mixture at a temperature between 20 and 35° C. over 3 to 6 hours. This affords the product in a rapidly filterable form. After treatment of the mother liquor with sodium hydroxide solution, it is possible to recover the base of the formula (IV) to an extent of about 95%.

The examples which follow illustrate the invention.

### EXAMPLE 1: PREPARATION OF 2-CHLORO-3-[(S)-METHYLSULFINYL]-4-(TRIFLUOROMETHYL)BENZOIC ACID

### Step 1: Preparation of 2-chloro-3-[(S,R)-methylsulfinyl]-4-(trifluoromethyl)benzoic Acid

A stirred 3 litre jacketed reactor is initially charged with 1 l of glacial acetic acid, and then 0.2 kg of 2-chloro-3-methylsulfanyl-4-(trifluoromethyl)benzoic acid is added. The cloudy mixture is heated to 60° C. and, at that temperature, a 35% aqueous hydrogen peroxide solution is added dropwise within 130 min and stirred at internal temperature 70° C. for 21 hours. The mixture is cooled to 20° C. and 100 ml of a 39% sodium hydrogensulfite solution is added dropwise. The mixture is then concentrated in a rotary evaporator down to a residual volume of about 20%. The residue is taken up in 1 l of water and alkalized with 120 ml of a 45% sodium hydroxide solution (pH 13-14). The aqueous solution is then washed with dichloromethane, and the aqueous phase removed is cooled to 5° C. and acidified with 280 ml of 32% hydrochloric acid. The product precipitates out as an oil and crystallizes after a few minutes. The solids are filtered off by cold filtration through a suction filter and washed with water and dried. 194 g of a beige solid is obtained.

HPLC (H₃PO₄): log P=0.96;

mass spectrometry: 287.0 (M+H)⁺, 328.1 (M+H+CH₃CN)⁺, 573.0 (2M+H)⁺:

¹H NMR [DMSO-D₆]: 14.2 (br s, 1H), 7.96-8.00 (m, 2H), 3.14 (s, 3H).

### Step 2: Preparation of 2-chloro-3-[(S,R)-methylsulfinyl]-4-(trifluoromethyl)benzoic Acid 2-Chloro-3-[(S)-methylsulfinyl]-4-(trifluoromethyl)benzoic acid [(1S)-1-(p-tolyl)ethyl]ammonium

In an inertized and stirred jacketed reactor. 1.06 kg of racemic 2-chloro-3-[(S,R)-methylsulfinyl]-4-(trifluoromethyl)benzoic acid is dissolved in 20 l of acetone and heated to 55° C. Under gentle reflux. 519.4 g of (S)-(−)-α,4-dimethylbenzylamine is added dropwise within four hours, and the resulting suspension is stirred at 52° C. overnight. The mixture is cooled down gradually to 20° C. within 6 hours. The suspension is filtered through a suction filter. The filtercake is then washed with acetone and subsequently dried at 40° C. under reduced pressure. This leaves 637 g of colourless crystals.

HPLC (H₃PO₄): log P=0.50/1.00;

mass spectrometry: 119.0 (amine-M+H)⁺, 286.9 (acid-M+H)⁺; chiral HPLC 95.1% ee;

¹H NMR [DMSO-D₆]: 8.23 (br s, 3H), 7.70-7.71 (m, 1H), 7.45-7.46 (m, 1H), 7.35-7.36 (m, 2H), 7.22-7.23 (m, 2H), 4.35 (q, 1H), 3.07 (s, 3H), 2.31 (s, 3H), 1.47 (d, 3H).

### Step 3: Preparation of 2-chloro-3-[(S)-methylsulfinyl]-4-(trifluoromethyl)benzoic Acid

A stirred jacketed reactor is initially charged with 4.9 l of ice-water, and 636 g of the salt from step 2 is suspended therein. Then a total of 0.55 l of a concentrated hydrochloric acid solution is added dropwise, and the temperature is kept between 0° C. and 5° C. The suspension is gradually warmed to room temperature and stirring is continued overnight. The suspension is then filtered through a suction filter. The filtercake is then washed with 3 l of distilled water and subsequently dried at 50° C. under reduced pressure. This leaves 408.5 g of colourless crystals.

HPLC (H₃PO₄): log P=1.00;

mass spectrometry: 286.9 (M+H)⁺; chiral HPLC 98.0% ee:

¹H NMR [DMSO-D₆]: 14.2 (br s, 1H) 7.96-7.99 (m, 2H), 3.14 (s, 3H).

### EXAMPLE 2: PREPARATION OF 2-CHLORO-N-(5-METHYL-1,3,4-OXADIAZOL-2-YL)-3-[((S)-METHYLSULFINYL)]-4-(TRIFLUOROMETHYL)BENZAMIDE

28.6 g (0.1 mol) of 2-chloro-3-[(S)-methylsulfinyl]-4-(trifluoromethyl)benzoic acid, 11 g (0.11 mol) of 2-amino-5-methyl-1,3,4-oxadiazole and 28.7 g (0.35 mol) of N-methylimidazole are dissolved in 200 ml of acetonitrile and stirred for 30 minutes. After cooling to 5° C., 18.9 g (0.16 mol) of thionyl chloride is added dropwise over 60 minutes such that the temperature remains between 5° C. and 10° C. This is followed by stirring at 20° C. for another 15 hours. The solvent is removed under reduced pressure, and water is added to the oily residue at 40° C. The product precipitates out and, after being filtered off, is washed with cold hydrochloric acid and water. After drying, 33.7 g (92%) of 2-chloro-N-(5-methyl-1,3,4-oxadiazol-2-yl)-3-[((S)-methylsulfinyl)]-4-(trifluoromethyl)benzamide with a melting point of 220° C. is obtained.

Optical rotation: (−)-69° (MeOH).

1. Chiral 3-sulfinylbenzoic acids of the respective absolute configuration given in formulae (I-R) and (I-S) 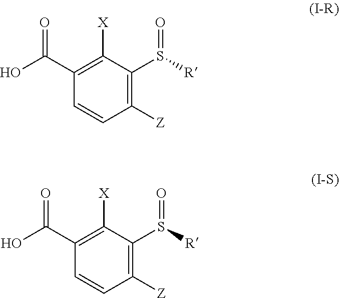
in which the substituents are defined as follows:
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR^{a}, S(O)ₙR^{b} or (C₁-C₆)-alkyl-OR^{a},
Z is halogen, (C₁-C₆)-alkyl, halo-(C₁-C₆)-alkyl, (C₃-C₆)-cycloalkyl or S(O)ₙR^{b},
R^{a} is (C₁-C₆)-alkyl or (C₃-C₆)-cycloalkyl,
R^{b} is (C₁-C₆)-alkyl or (C₃-C₆)-cycloalkyl,
n is 0, 1 or 2.

2. 3-Sulfinylbenzoic acids according to claim 1, in which
X is F, Cl, Br, methyl, ethyl, i-Pr, c-Pr, OMe, SMe, SEt, CH₂OMe or CF₃,
R′ is methyl, ethyl, c-Pr, CH₂-cPr, CH₂CH₂OMe, c-Pr, CH₂-cPr or CH₂CH₂OMe,
Z is F, Cl, Br, I, methyl, ethyl, c-Pr, i-Pr, SMe, S(O)Me, S(O)₂Me, S(O)₂Et, CF₃, C₂F₅ or CHF₂.

3. 3-Sulfinylbenzoic acids according to claim 1, in which
X is F, Cl, Br, methyl, ethyl, c-Pr, OMe, SMe, SEt, CH₂OMe or CF₃,
R is Me, Et, c-Pr, CH₂-cPr or CH₂CH₂OMe,
Z is Cl, Br, methyl, ethyl, c-Pr, i-Pr, S(O)₂Me, S(O)₂Et, CF₃, C₂F₅ or CHF₂.

4. 3-Sulfinylbenzoic acids according to claim 1, in which
X is Cl or methyl,
R is methyl or c-Pr,
Z is CF₃ or CHF₂.

5. 3-Sulfinylbenzoic acids according to claim 1 with an enantiomeric excess (ee) of at least 94%.

6. 3-Sulfinylbenzoic acids according to claim 5 with an enantiomeric excess (ee) of at least 99%.

7. Process for preparing 3-sulfinylbenzoic acids according to claim 1, characterized in that
a) racemic compounds of the formula (I-rac) are reacted with an enantiomerically pure amine of the general formula (II),
b) one of the two crystallized diastereomeric salts (III-dr) or (III-ds) is filtered off, purified and released by addition of water and acid to give the 3-sulfinylbenzoic acid of the formula (I-R) or (I-S),
c) the other diastereomeric salt from the mother liquor of step a) is released by addition of water and acid to give the 3-sulfinylbenzoic acid of the formula (I-R) or (I-S), and
d) in which, in formula (II),
R¹ is methyl, ethyl, n-propyl, isopropyl, n-butyl, isobutyl, and
R² is hydroxymethyl, phenyl, 4-methylphenyl:

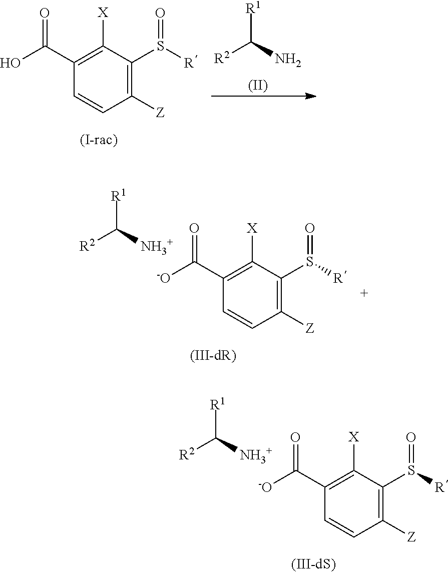

8. Process for preparing N-(1,3,4-oxadiazol-2-yl)phenylcarboxamides having the absolute configuration given in formula (I*) by reacting 2-amino-1,3,4-oxadiazoles of the general formula (V) with inventive 3-sulfinylbenzoic acids of the general formula (I-S), 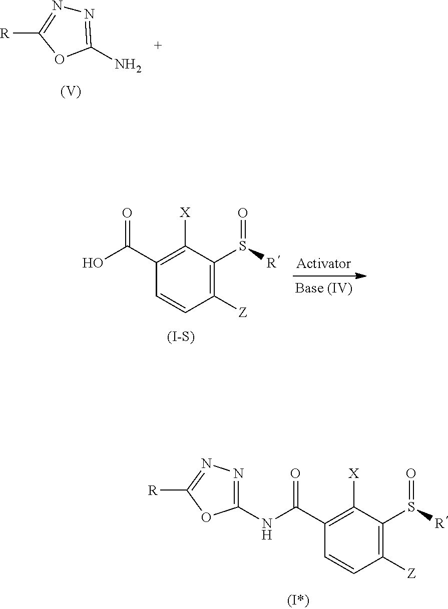
characterized in that it is performed
a) in the presence of an activating reagent (activator) from the group consisting of thionyl chloride, phosgene, diphosgene, mesyl chloride, tosyl chloride, POCl₃, PCl₅, oxalyl chloride and C₁-C₈-alkyl-OC(O)Cl, and
b) in the presence of a base of the general formula (IV), 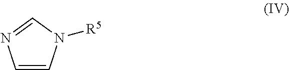
and
c) in which the substituents are as defined below:
R is hydrogen, (C₁-C₆)-alkyl, (C₃-C₇)-cycloalkyl, methoxymethyl or methoxyethyl,
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR¹, S(O)ₙR² or (C₁-C₆)-alkyl-OR¹,
Z is halogen, (C₁-C₆)-alkyl, halo-(C₁-C₆)-alkyl, (C₃-C₆)-cycloalkyl or S(O)ₙR²,
R¹ is (C₁-C₆)-alkyl or (C₃-C₆)-cycloalkyl,
R² is (C₁-C₆)-alkyl or (C₃-C₆)-cycloalkyl,
R⁵ is C₁-C₁₂-alkyl or phenyl,
n is 0, 1 or 2.

9. Process for preparing N-(1,3,4-oxadiazol-2-yl)phenylcarboxamides having the absolute configuration given in formula (I**) by reacting 2-amino-1,3,4-oxadiazoles of the general formula (V) with inventive 3-sulfinylbenzoic acids of the general formula (I-R), 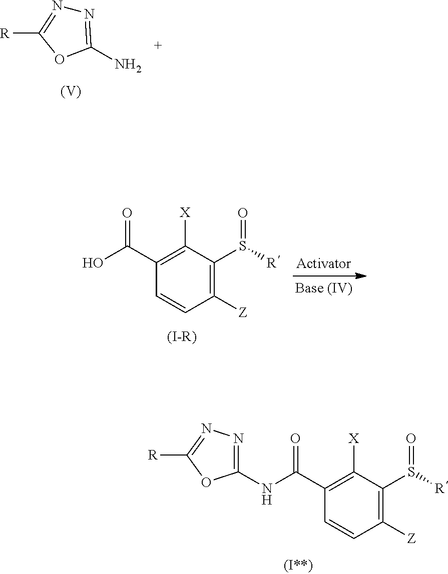
characterized in that it is performed
a) in the presence of an activating reagent (activator) from the group consisting of thionyl chloride, phosgene, diphosgene, mesyl chloride, tosyl chloride, POCl₃, PCl₅, oxalyl chloride and C₁-C₈-alkyl-OC(O)Cl, and
b) in the presence of a base of the general formula (IV), 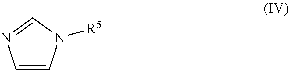
and
c) in which the substituents are as defined below:
R is hydrogen, (C₁-C₆)-alkyl, (C₃-C₇)-cycloalkyl, methoxymethyl or methoxyethyl,
R′ is (C₁-C₆)-alkyl, (C₃-C₆)-cycloalkyl, (C₁-C₆)-alkyl-O—(C₁-C₆)-alkyl or (C₃-C₆)-cycloalkyl-(C₁-C₆)-alkyl,
X is halogen, (C₁-C₆)-alkyl, halo-(C₁-C₆)-alkyl, (C₃-C₆)-cycloalkyl, OR¹, S(O)ₙR² or (C₁-C₆)-alkyl-OR¹,
Z is halogen, (C₁-C₆)-alkyl, halo-(C₁-C₆)-alkyl, (C₃-C₆)-cycloalkyl or S(O)ₙR²,
R¹ is (C₁-C₆)-alkyl or (C₃-C₆)-cycloalkyl,
R² is (C₁-C₆)-alkyl or (C₃-C₆)-cycloalkyl,
R⁵ is C₁-C₁₂-alkyl or phenyl,
n is 0, 1 or 2.

10. Process according to claim 8, in which
R is hydrogen or methyl,
X is F, Cl, Br, methyl, ethyl, i-Pr, c-Pr, OMe, SMe, SEt, CH₂OMe or CF₃,
R is methyl, ethyl, c-Pr, CH₂-cPr, CH₂CH₂OMe, c-Pr, CH₂-cPr or CH₂CH₂OMe,
Z is F, Cl, Br, I, methyl, ethyl, c-Pr, i-Pr, SMe, S(O)Me, S(O)₂Me, S(O)₂Et, CF₃, C₂F₅ or CHF₂.

11. Process according to claim 8, in which
R is hydrogen or methyl,
X is F, Cl, Br, methyl, ethyl, c-Pr, OMe, SMe, SEt, CH₂OMe or CF₃,
R is Me, Et, c-Pr, CH₂-cPr or CH₂CH₂OMe,
Z is Cl, Br, methyl, ethyl, c-Pr, i-Pr, S(O)₂Me, S(O)₂Et, CF₃, C₂F₅ or CHF₂.

12. Process according to claim 8, in which
R is hydrogen or methyl,
X is Cl or methyl,
R is methyl or c-Pr,
Z is CF₃ or CHF₂.

13. Process according to claim 8, in which the compounds of the formulae (V) and (I-S) or (V) and (I-R) are used in a molar ratio of 0.8 to 1.5.

14. Process according to claim 8, in which the activator is selected from the group consisting of thionyl chloride, phosgene, diphosgene, mesyl chloride, tosyl chloride, POCl₃, PCl₅, oxalyl chloride and C₁-C₈-alkyl-OC(O)Cl, and this activator and the compounds of the formula (I-S) or (I-R) are used in a molar ratio of 1 to 2.

15. Process according to claim 8, in which the activator is selected from the group consisting of thionyl chloride, phosgene and diphosgene.
